(19) United States
(12) Patent Application Publication
(10) Pub. No.: US 2011/0257417 A1
(43) Pub. Date: Oct. 20, 2011

(54) HIV-1 INHIBITING PHARMACEUTICAL COMPOSITION CONTAINING AN ECKLONIA CAVA-DERIVED PHLOROGLUCINOL POLYMER COMPOUND

(75) Inventors: Moon-Moo Kim, Busan (KR); Se-Kwon Kim, Busan (KR); Sang-Hoon Lee, Busan (KR)

(73) Assignee: Pukyong National University Industry-Academic Cooperation Foundation, Busan (KR)

(21) Appl. No.: 13/123,206

(22) PCT Filed: Sep. 21, 2009
(86) PCT No.: PCT/KR2009/005354

§ 371 Date: Jul. 1, 2011

(30) Foreign Application Priority Data
Oct. 7, 2008 (KR) 1020080098327

(51) Int. Cl. C07D 407/10

(52) U.S. Cl. 549/361

(57) ABSTRACT

The present invention relates to a HIV-1 inhibiting pharmaceutical composition containing, as an active ingredient, 6,6′-bieckol which is a phloroglucinol polymer compound separated from Ecklonia cava. The Ecklonia cava-derived 6,6′-bieckol according to the present invention inhibits HIV-1 induced cell fusion, the cell lysis effect, and the cytopathogenic effect including virus p24 antibody production, exhibits RT enzyme inhibition activity and HIV-1 infection inhibition activity, and shows no cytotoxicity at the concentration that almost perfectly inhibits HIC-1 replication.

### TECHNICAL FIELD

The present invention relates to a HIV-1 inhibiting pharmaceutical composition containing an Ecklonia cava-derived phloroglucinol polymer compound. More particularly, the present invention relates to a HIV-1 inhibiting pharmaceutical composition containing 6,6′-bieckol as an active ingredient which is a phloroglucinol polymer compound separated from Ecklonia cava.

### BACKGROUND ART

Human immunodeficiency virus type-1 (Human Immunodeficiency virus type-1; HIV-1) is identified as the causative agent of acquired immunodeficiency syndrome (AIDS) which is one of the most important diseases with about 33 million people infected worldwide up to now. In 2007, 2.1 million people died of AIDS related diseases and 2.5 million people were newly infected with HIV. According to UNAIDS estimations, every day 6800 people become newly infected with HIV and 5700 people die of AIDS and its related disorders. However, a mechanism by which the virus depletes the immune system is not fully identified.

After AIDS was known to the public in early 1980s, the first generation anti-HIV drugs have been developed as the promising agents to cure AIDS patients. However, failure in anti-AIDS treatment was observed in more than 50% of the patients infected with HIV because of drug-resistant strains of virus. Accordingly, the need for potential candidates showing higher inhibitory activity against various HIV strains including the ones which are resistant to drugs currently used for antiretroviral therapy is increasing.

Recently, there are significant advances in rational drug design and highly active compounds have been synthesized. However, natural occurring products are still known as the richest source of bioactive leading compounds, as well as their derivatives.

In this regard, natural products are great sources for the development of new generation anti-HIV drugs which are more effective with less side-effect. So far, numerous compounds isolated from natural resources have been found to exhibit significant anti-HIV activity. Considerable evidences have emerged not only from the research and publications, but also from controlled clinical studies of natural product-derived substances as important leads for the development of antiviral drugs against viral infections caused by HIV. Many compounds which are originated from plant and inhibit HIV during various stages of HIV's life cycle have been described, for example, alkaloids, coumarins, carbohydrates, flavonoids, lignans, phenolics, quinines, phospholipids, terpenes and tannins. Unlike terrestrial natural products which have been known as traditional medicine sources for hundreds of years, the intensive research on marine natural products started in the middle of the last century. Since the oceans cover more than 70% of surface of the earth, they represent an enormous reservoir for the discovery of novel therapeutic agents. Because of such a big potential, natural product research has increasingly turned to marine natural products, and some of them are currently in a stage of clinical or preclinical evaluation. Marine organisms are among the leading sources of anti-HIV natural products. Cyanovirin-N, an 11-kDa protein from cyanobacteria, has been investigated on its antiviral properties and it is found that Cyanovirin-N irreversibly inactivates HIV through its high affinity to gp120. Various sulfated polysaccharides, most of which have high HIV inhibitory activity at the viral entry stage, have been isolated from marine algae. Dextran sulfate, carrageenans, galactan sulfate and xylomannan are among the leading marine-based compounds with anti-HIV activity.

Tannins are naturally occurring water-soluble polyphenolic compounds, which show their HIV-1 inhibitory action probably by inhibiting polymerase and ribonuclease activities of HIV-1 RT. Gallotannins, geranium and corilagin inhibit HIV-1 replication in MT-4 cells. Geraniim effectively blocked viral entry and inhibited RT activity of HIV-1 at an IC₅₀ of 1.9 μM. 1,3,4-tri-O-galloylquinic acid and 3,5-di-O-galloylshikimic acid of tannin derivatives inhibit virus-cell interactions via tight binding to virions, inactivation of them and prevention of infection. Phlorotannins are one of tannin derivatives, which are composed of several phloroglucinol units linked to each other in different ways and mostly isolated from red and brown algae. It has been reported that phlorotannins have antioxidant, anti-inflammatory, antibacterial and anti-MMP activities. Ahn et al. insisted that 8,8′-bieckol and 8′,4′″-dieckol of phloroglucinol derivatives inhibit the activity of recombinant RT and protease of HIV-1 in vitro.

Ecklonia cava is edible brown algae and found in the ocean off Japan and Korea. Although it has been abundantly produced in Jeju Island of Korea (30,000 ton/year), it is not consumed in Europe at all. Such valuable brown algae are used as food stuff, animal feedstuff, fertilizer and folk medicine for gynecopathy.

Specifically, Ecklonia cava is used as an active ingredient in a skin-whitening cosmetic composition (Korean Patent Application No. 10-2000-0048933), an anti-wrinkle cosmetic composition (Korean Patent Application No. 10-2001-0025580) and a cosmetic composition for pimpled skin (Korean Patent Application No. 10-2006-0093939) in a food or cosmetic industry.

Further, as to usage of phloroglucinol polymer compounds derived from Ecklonia cava, Korean Patent Application No, 10-2002-83555 entitled ‘Composition for a degenerative arthritis comprising a seaweed extract’ discloses a composition for preventing and improving degenerative arthritis comprising a seaweed extract having more than 10% of polyphloroglucinol polymer complexes comprised of 15 polyphenols extracted from seaweed including Ecklonia cava. And, Korean Patent Application 10-2004-3912 entitled ‘Composition for improvement of cardiovascular disease’ discloses a composition for improving cardiovascular diseases, containing alginic acid, fucoidan, laminarin, phloroglucinol polymers and fucosterol derived from seaweeds including Ecklonia cava.

Further, Korean Patent No. 10-0518179 entitled ‘Composition for improvement of sexual function’ discloses a composition for improving sexual function, comprised of extract powders of seaweeds including Ecklonia Cava wherein phloroglucinol polymer compounds, a strong antioxidant, protecting vascular endothelial cells from active oxygen species and fucosterol component adjusting ACE (Angiotensin Converting Enzyme) known as causing vasoconstriction to interfere with erection are included to maintain the relaxation state of blood vessel muscles, and various vegetable materials supplying nutrients essential for vigor and sexual function. Also, it discloses that the composition does not have side effects and exhibits remarkable effects on improvement of sexual function.

As a result of our continuous research to discover the bioactive natural products from marine sources, the present inventors could reach the present invention by obtaining a series of phloroglucinol derivatives from marine brown algae EC, characterizing them through physicochemical and NMR spectroscopic methods and identifying that 6,6′-bieckol, one of phloroglucinol polymer derivatives, has inhibition activity against HIV-1 among the isolated phlorotannins for the first time.

### DETAILED DESCRIPTION

### [Technical Problem]

The present invention aims at providing a HIV-inhibiting pharmaceutical composition containing phloroglucinol polymer compounds derived from Ecklonia cava as an active ingredient.

### [Technical Solution]

The above purpose of the present invention was achieved by separating phloroglucinol polymer compounds from Ecklonia cava and discovering that among the separated compounds, 6,6′-bieckol inhibited the HIV-1 cytopathic effects including HIV-1 induced syncytia formation, lytic effects and viral p24 antigen production and exhibited RT enzyme inhibitory and HIV-1 entry activity in addition to its other biological properties, and unlike most of other tannins, 6,6′-bieckol exhibited no cytotoxicity at a concentration which inhibited HIV-1 replication almost completely.

Therefore, the present invention provides a HIV-1 inhibiting pharmaceutical composition containing phloroglucinol polymer compounds from Ecklonia cava as an active ingredient.

Phloroglucinol polymer compounds according to the present invention is preferably 6,6′-bieckol.

In this specification, a term ‘active ingredient’ means a material or materials group (including crude drugs whose pharmacological active ingredients are not identified) which are expected to exhibit efficacy or effect of a medicine directly and indirectly through its intrinsic pharmacological action.

According to the present invention, 6,6′-bieckol exhibits no cytotoxicity up to 500 μM and has the CC₅₀ value of 484 μM (FIG. 1A). HIV-1 induced cytopathic effect was measured by quantification of syncytia formation and the lytic effect of virus on C8166 and CEM-SS cells, respectively. 6,6′-bieckol inhibits HIV-1 induced syncytia formation and protects the cells from lytic effect of virus in a dose-dependent manner. EC₅₀ of 6,6′-bieckol on the inhibition of the HIV-1 induced syncytia formation is 1.72 μM (FIG. 1B). At the highest concentration of 6,6′-bieckol (25 μM), 88% of syncytia formation is inhibited. It is found that 6,6′-bieckol protects cells from HIV-1 induced lytic effects in vitro. With treatment at the highest concentration of 6,6′-bieckol (25 μM), the rate of protection of infected cells is more than 96% (FIG. 1C). The therapeutic index (TI) of 6,6′-bieckol is around 393. Inhibition of HIV-1 induced cytopathic effect is parallel with those of p24 antigen production. EC₅₀ of 6,6′-bieckol on inhibiting HIV-1 p24 antigen production is 1.26 μM (FIG. 1D). The inhibitory effect of 6,6′-bieckol on HIV-1 p24 antigen production was further investigated by Western blot analysis of cell and culture supernatant (FIG. 2A). HIV-1 RT enzyme from cell culture supernatant is strongly inhibited by 6,6′-bieckol at relatively low Concentrations (50.46% inhibition at 1 μM). The rate of inhibition of HIV-1 RT enzyme activity by 6,6′-bieckol at 10 μM is 96.33% (FIG. 1C).

According to the data obtained from co-cultivation of C8166 cells with H9 cells chronically infected with HIV-1IIIB, 6,6′-bieckol inhibits the entry of HIV-1. 6,6′-bieckol inhibits cell-virus fusion(FIG. 1B) and cell-cell fusion(FIG. 3A). The result of co-cultivation assay shows that the inhibition rate of syncytia formation is around 80% with treatment at the highest concentration of 6,6′-bieckol.

In the present invention, the anti-HIV-1 activity of 6,6′-bieckol was investigated. It is found that 6,6′-bieckol inhibits the cytopathic effects of HIV-1 on CEM-SS and C8166 cells as well as the activity of HIV-1 reverse transcriptase enzyme activity in vitro. Phloroglucinol derivatives exhibit HIV-1 inhibitory activity mostly by blocking the interaction between RT and RNA template.

In FIGS. 1B and 3B, data obtained from HIV-1 induced cytopathic effects and RT activity studies indicate that HIV-1 replication inhibition correlates with the inhibition of reverse transcriptase. 6,6′-bieckol inhibits both HIV-1 induced lytic effect and HIV-1 RT activity almost completely. These data reveal that unlike other tannins, 6,6′-bieckol is non-cytotoxic at a concentration which significantly inhibits HIV-1 replication and reverse transcriptase activity.

Further, the inhibitory effect of 6,6′-bieckol on HIV-1 entry was investigated. According to the result of data obtained from co-cultivation of uninfected C8166 cells with H9/HIV-1IIIB cells, 6,6′-bieckol inhibits the HIV-1 induced syncytia formation. Tannins show their inhibitory activity on HIV-1 entry by inhibiting gp416 six-helix bundle formation. However, when entry inhibition profile of 6,6′-bieckol is compared with the inhibition of cytopathic effects, it could be concluded that HIV-1 induced cytopathic effect is reduced not only by the entry inhibition, but also by subsequent inhibition of HIV-1 reverse transcriptase enzyme activity.

The present inventors determined production of p24 antigen by p24 antigen capture ELISA and Western blot analysis. According to the data of the ELISA (FIG. 1D), 6,6′-bieckol inhibits the production of p24 antigen more than 90% at 10 μM. As shown in FIG. 2C, the inhibition of p24 antigen production by 6,6′-bieckol at a concentration of 25 μM was comparable to that of AZT (5 μM). 8,8′-bieckol, which is structurally similar to 6,6′-bieckol, inhibits HIV-1 recombinant RT and protease activity with IC₅₀ of 0.51 μM and 81.5 μM, respectively. Reverse transcriptase inhibitory activity of 6,6′-bieckol is consistent with that of 8,8′-bieckol.

According to the Western blot analysis, no or weak bands were detected at the locations of p55 gag related proteins (p55, p41, and p24) in addition to p24, which indicates that 6,6′-bieckol can not inhibit the protease. However, 6,6′-bieckol inhibits the p24 antigen production in a dose-dependent manner both in culture supernatant and cell (FIGS. 2A and 2B) by inhibiting the activity of reverse transcriptase. Generally, the structure-activity relationships should go to the unique skeleton with dibenzodioxin linkage, linkage positions of phloroglucinols, and phenolic groups exiting in 6,6′-bieckol.

Based on the above, 6,6′-bieckol according to the present invention is a novel safe compound of natural origin with significant inhibitory activity of HIV-1 replication and reverse transcriptase. Accordingly, 6,6′-bieckol according to the present invention can be usefully used for designing novel HIV-1 RT inhibitors with its special structure.

[Advantageous Effects]6,6′-bieckol derived from Ecklonia Cava according to the present invention inhibits the cytopathic effects of HIV-1 including HIV-1 induced syncytia formation, lytic effects, and viral p24 antigen production and exhibits RT enzyme inhibitory activity and HIV-1 entry inhibitory activity. Further, 6,6-bieckol exhibits no cytotoxicity at a concentration which inhibits HIV-1 replication almost completely.

### DESCRIPTION OF DRAWINGS

FIG. 1 shows cytotoxicity and HIV-1 inhibitory activity of 6,6′-bieckol according to the present invention. Values represent means ±SE (n=3). (A) Cytotoxicity of 6,6′-bieckol on CEM-SS cells measured by MIT assay, (B) Inhibition of HIV-1 induced syncytia formation determined using a microscope, (C) Protection of CEM-SS cells from HIV-1 induced lytic effects measured MTT assay, and (D) Inhibition of p24 antigen production measured by ELISA.

FIG. 2 shows effect of 6,6′-bieckol on viral p24 protein production in HIV-1 infected cells. H9 cells were acutely infected with HIV-1_{IIIB} and treated with 6,6′-bieckol at 0.1, 0.25, 1, 2.5, 10 and 25 μM or AZT (5 μM) for 4 days and then cells (A) and supernatants (B) were analyzed by Western blot analysis. Areas and intensities of protein bands were measured by densitometry and expressed as a percentage of p24 expression compared to protein level of infected-untreated cells (C). Values represent means ±SE (n=3).

FIG. 3 shows effect of 6,6′-bieckol on HIV-1 entry and inhibition of HIV-1 RT activity. Azidothymidine (AZT) (5 μM) and dextran sulfate (DS) (100 μg/ml) were used as positive controls, respectively. Values represent means ±SE (n=3). (A) HIV-1 entry was measured by quantification of fusion between normal C8166 and H9/HIV-1_{IIIB} cells. (B) The inhibitory effect of 6,6′-bieckol on HIV-1 RT activity was determined by fluorescent RT activity assay.

### MODE FOR INVENTION

The present invention will be described in detail herein below with reference to working examples. It should be also clearly understood that the working examples are made only for illustration and the present invention is not limited to the working examples.

### WORKING EXAMPLES

¹H NMR (400 MHz) and ¹³C NMR (100 MHz) spectra were recorded on a JEOL JNM-ECP 400 NMR spectrometer (JEOL, Japan), using DMSO-d₆ solvent peak (d 2.50 ppm in ¹H and d 39.5 ppm in ¹³C NMR) as an internal reference standard. Some signals are represented as the approximated third decimal place. Although such representation is to distinguish signals having very close values, but the signals could be clearly distinguished by visual inspection of the spectra.

MS spectra were obtained on a JEOL JMS-700 spectrometer (JEOL, Japan). Extraction of EC was performed using Extraction Unit (Dongwon Scientific Co., Korea). Column chromatography was carried out by silica gel 60(230-400 mesh, Merck, Germany) and Sephadex LH-20 (Sigma, St.Louis, Mo., USA). TLC was run on precoated Merck Kieselgel 60 F254 plates (0.25 mm) and the spots on the TLC plate were detected under UV lamp (254 and 365 nm) using CHCl₃/MeOH/H₂O/acetic acid (65:25:4:3, v/v) as development solvent system, and Vanillin-I H₂SO₄ as the detecting agent for phenolic compounds. All the solvents for column chromatography were of a reagent grade from commercial sources.

AZT, dextran sulfate, and polyethylene glycol (Mw 8000 Da) were obtained from Sigma Chemical Co. (St. Louis, Mo., USA). Primary and secondary antibodies for Western blot were purchased from SantaCruz Biotechnology (CA, USA). HIV-1 p24 antigen capture ELISA kit was obtained from Perkin-Elmer Life Sciences (Boston, Mass., USA). Reverse transcriptase activity assay kit was purchased from InvitroGen (CA, USA). Cell culture medium (RPMI 1640), penicillin/streptomycin, fetal bovine serum (FBS), and other cell culture materials were obtained from Gibco BRL, Life Technology (NY, USA) and Sigma Chemical Co. (St. Louis, Mo., USA).

H9 and H9/HIV-1IIIB cell lines were obtained through American Type of Culture Collection (Manassas, Va., USA). CEM-SS cell lines from Dr. P. Nara and C8166 cell lines from Dr. G. Farrar were provided by the EU Programme EVA Centre for AIDS Reagents, NIBSC, UK. CEM-SS, C8166, H9 and H9/HIV-1IIIB cell lines were grown in RPMI 1640 medium supplemented with 10% FBS, 100 μg of streptomycin per ml and 100 U of penicillin per ml. HIV-1IIIB virus stock was obtained from the culture supernatant of chronically infected H9/HIV-1IIIB cells. Cell-free virus was harvested from the supernatant by centrifugation and filtration through 0.22 μm filter. The virus stocks were stored as small aliquots at 80° C. until use.

All the experiments were performed in triplicate and the obtained values represent average ±SD.

### Example 1

### Extraction and Purification of Phloroglucinol Derivative from Ecklonia Cave (EC)

The marine edible brown seaweed, EC, was collected from Jeju Island coast of Korea from October 2004 to March 2005. Fresh EC was washed three times with water to remove salt. The lyophilized EC was ground into powder before extraction. The dried EC powder (10 kg) was extracted using an extraction unit with MeOH (35 L) for 10 days under stirring. The methanol extract (273 g) was suspended in water and partitioned with n-hexane (35.92 g), CH₂Cl₂ (20.49 g), EtOAc (24.87 g) and n-BuOH, in sequence. The EtOAc fraction (24.87 g), which exhibited the most potent anti-HIV activity on H9 and H9/HIV-1_{IIIB} human cells, was subjected to a silica gel flash chromatography and eluted with a gradient solvent system of Hexane/EtOAc/MeOH to yield ten subfractions of F1 to F10.

The F5 fraction (378.39 mg) with the highest activity on anti-HIV was further purified by Sephadex LH-20 with only MeOH to afford the phloroglucinol derivative, compound 1 (102.85 mg) as light brown amorphous powder.

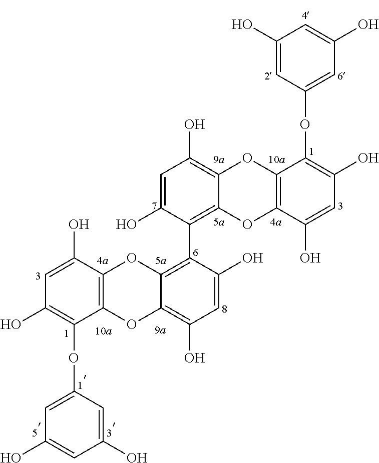

The molecular formula of compound 1 was determined as C₃₆H₂₂O₁₈ from LREIMS, ¹H, ¹³C, and ¹³C DEPT spectrum data. The ¹H NMR spectrum of compound 1 showed two AB₂ systems at d 5.75 (2H,J=2.2 Hz), and 5.80 (1H, J=2.2 Hz), and two singlet signals at d 6.05 (2H, s) and 6.09 (2H, s) as well as 12 phenolic OH protons at d 9.29 (2H, s), 9.16 (4H,s), 9.15 (2H, s), 9.09 (2H, s) and 8.65 (2H,s), respectively.

The ¹³C NMR spectrum revealed the presence of 10 unsubstituted and 24 O-bearing aromatic carbons, and two quaternary aromatic carbon signals. The molecular weight of compound 1 was twice more than bieckol due to extra two phloroglucinol units without two protons loss resulting from the direct linkage (742 vs 372). These data described above clearly indicated that compound 1 was composed of six phloroglucinol units. On the basis of the above data and an exact comparison with those values in the literature, compound 1 was determined as 6,6′-bieckol having molecular formula 1. The 6.4 ppm difference of 13C chemical shift value at C-8 between 6,6′-bieckol and 8,8′-bieckol led to the clear classification of both similar phlorotannins.

Compound 1: light brown powder (lyophilized); 1H NMR (DMSO-d6, 400 MHz) d 9.29 (1H, s, OH-9), 9.16 (2H,s, OH-30, 50), 9.15 (1H, s, OH-2), 9.09 (1H, s, OH-4), 8.65 (1H,s, OH-7), 6.09 (1H, s, H-3), 6.05 (1H, s, H-8), 5.80 (1H, d, J=2.2 Hz, H-40), 5.75 (2H, d, J=2.2 Hz, H-20, 60); 13C NMR (DMSO-d6, 100 MHz) d 123.5 (s, C-1)), 145.4 (s, C-2), 97.7(d, C-3), 141.4(s, C-4) 121.9(s, C-4a), 141.3(s, C-5a), 99.7 (d, C-6), 151.3 (s, C-7), 97.8 (d, C-8), 144.5 (s,C-9), 122.7 (s, C-9a), 137.2 (s,C-10a), 160.4 (s, C-10), 93.7 (d, C-20), 158.8 (s, C-30), 96.1 (d, C-40), 158.8 (s, C-50), 93.7 (d, C-60); LREIMS m/z 742.10[M+].

### Example 2

### Cell Viability Assay

The cytotoxic concentrations of 6,6′-bieckol were determined by MTT assay, which is a method based on the reduction of 3-(4,5-dimethylthiazol-2-yl)-2,5-diphenyltetrazolium bromide (MTT). 400 μL of medium containing CEM-SS or H9 cells were cultured in a 48-well plate at a density of 10⁵ cells/ml. The plate was incubated overnight and treated with 100 φl of RPMI 1640 medium with/without 6,6′-bieckol. After 72 h of incubation, 100 μl of 500 μg/ml MTT was added to each of the wells and the plate was incubated for another 4 h at 37° C. The blue formazan was dissolved in acidified propanol containing 50% DMSO and 4% Triton X-100. Optical density was measured at 540 nm with a microplate reader. The optical density of formazan formed by untreated cells was taken as 100% of viability.

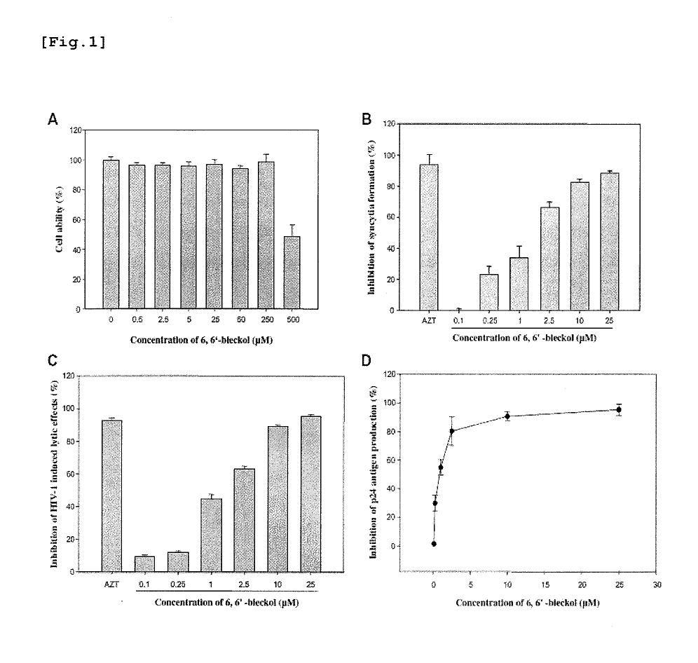

6,6′-bieckol didn't show cytotoxicity up to 500 μM and had CC₅₀ value of 484 μM (FIG. 1).

### Example 3

### Measurement of Anti-HIV Activity

### Example 3-1: Inhibition of Syncytia Formation

1×10⁵ C8166 cells in aliquots of 300 μl were seeded in triplicate to a 48-well plate containing 100 μl of serial dilutions of the compound in complete medium. After 2 h of incubation, the cells were infected with 100 μl of stock supernatant of HIV-1IIIB diluted in complete medium at 200 CCID₅₀. The plates were incubated at 37° C. for 72 h and the number of syncytia formation was determined microscopically.

HIV-1 induced cytopathic effect was measured by quantification of syncytia formation and the lytic effect of virus on C8166 and CEM-SS cells, respectively. 6,6′-bieckol inhibited HIV-1 induced syncytia formation and protected the cells from lytic effect of virus in a dose dependent manner. EC₅₀ of 6,6′-bieckol on the inhibition of the HIV-1 induced syncytia formation was 1.72 μM (FIG. 1B). With the highest concentration of 6,6′-bieckol (25 μM), 88% of syncytia formation was inhibited.

### Example 3-2: Inhibition of Lytic Effects of HIV-1

In order to determine the anti-HIV-1 activity of 6,6′-bieckol on acutely infected CEM-SS cells, an MTT-formazan-based assay was used. Cells in log-growth phase were washed and resuspended in a complete medium, and a 300 μl aliquot containing 1×10⁵ cells was added in three portions to the wells of a 48-well plate containing the dilutions of the compound in a volume of 100 μl of medium. Stock supernatants of HIV-1IIIB were diluted in a complete medium to yield sufficient cytopathicity (90% cell kill in 7 days), and a 100 μl aliquot was added to the wells. Plates were incubated for 7 days at 37° C. and cell viability was determined on the 7^{th} day by MTT method as described before.

6,6′-bieckol protected the cells from HIV-1 induced lytic effects in vitro. With treatment at highest concentration of 6,6′-bieckol (25 μM), more than 96% of the infected cells was protected (FIG. 1C). The therapeutic index (TI) of 6,6′-bieckol was around 393. Inhibition of HIV-1 induced cytopathic effect was parallel with that of p24 antigen production. EC₅₀ of 6,6′-bieckol on inhibiting HIV-1 p24 antigen production was 1.26 1M (FIG. 1D).

### Example 3-3: Western Blot Analysis

1×10⁶ cells/ml H9 cells were cultured in cell culture plates. Following sample treatment, plates were incubated for 2 h and infected with HIV-1IIIB at 200 CCID₅₀. After 96 h of incubation at 37° C., cells were pelleted at 1000 rpm for 10 mM and supernatant was harvested. The cells were washed 3 times with PBS and lysed with 500 μl of lysis buffer containing 50 mM Tris-HCl (pH 7.5), 0.4% Nonidet P-40, 120 mM NaCl, 1.5 mM MgCl₂, 2 mM phenylmethylsulfonyl fluoride, 80 μg/ml leupeptin, 3 mM NaF and 1 mM DTT. Hundred micrograms of total protein was used for immunoblot analysis.

Culture supernatant containing virus was filtered through 0.22 μm filter, mixed with 30% polyethylene glycol (PEG) (50% v/v) with 0.4 M NaCl, and virus particles were pelleted at 15000 rpm for 45 min. The viral pellets were lysed, resuspended in SDS sample buffer and equal volume of viral lysates (20 μl) was loaded onto SDS gel. The proteins were subjected to denaturating SDS-PAGE in 25 mM Tris, 192 mM glycine, 0.1% SDS with a 4% stacking, and 10% separating gel. Separated proteins were transferred onto a nitrocellulose membrane and blocked in Tris buffered saline containing 0.1% (v/v) Tween 20 (TBS-T) and 5% skim milk powder. The membrane was probed with mouse anti-p24 monoclonal antibody and horseradish peroxidase (HRP)-conjugated anti-mouse IgG secondary antibody (1:5000, SantaCruz). The proteins were visualized by chemiluminescence (Fujifilm Life Science, Tokyo, Japan).

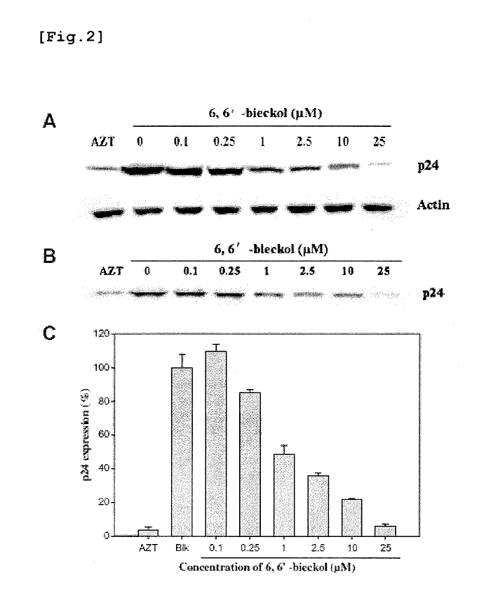

The inhibitory effects of 6,6′-bieckol on HIV-1 p24 antigen production were further determined by Western blot analysis of cell and culture supernatant (FIG. 2A). HIV-1 RT enzyme from cell culture supernatant was strongly inhibited by 6,6′-bieckol at relatively low concentrations (50.46% inhibition at 1 μM). 96.33% of HIV-1 RT enzyme activity was inhibited by 6,6′-bieckol at 10 μM (FIG. 1C).

Example 3-4: Reverse Transcriptase Activity assay

The activity of HIV-1 reverse transcriptase in the viral lysate was evaluated using a fluorescence RT assay kit (InvitroGen) according to the manufacturer's protocol. Briefly, 20 μl of reaction mixture containing a template/primer hybrid, poly(A)/d(T)16, and dTTP as a triphosphate substrate was added to the wells of a microtiter plate and mixed with 5₁x1 of viral lysate containing various concentrations of 6,6′-bieckol. After incubation at 37° C. for 1 h, the reaction was stopped by the addition of 2 μl of 200 mM EDTA to each reaction. Fluorescence intensity was measured at 480 nm (excitation) and 520 nm (emission) with a GENiosmicroplate reader (Tecan Austria GmbH, Austria) after the addition of 173 μl of fluorescent PicoGreenreagent, which selectively binds to sDNA or DNA-RNA heteroduplexes over single-stranded nucleic acids or free nucleotides.

In the present invention, the anti-HIV-1 activity of 6,6′-bieckol was investigated. 6,6′-bieckol was shown to inhibit the cytopathic effects of HIV-1 on CEM-SS and C8166 cells as well as the activity of HIV-1 reverse transcriptase enzyme activity in vitro. Phloroglucinol derivatives mostly exhibit HIV-1 inhibitory activity by blocking the interaction between RT and RNA template. This type of inhibition is similar to those of some flavonoids. Kilkuskie et al. reported that among the tested tannins, most of them were cytotoxic at the effective concentration, which means therapeutic indices (TI) of the compounds were around 1. The HIV replication and reverse transcriptase inhibitory activities of a variety of tannins including gallotannins, ellagitannins, condensed and complex tannins were evaluated in order to investigate the correlation between HIV inhibitory activity and RT inhibitory activity of tannins. The results indicated that reverse transcriptase inhibition did not correlate with the inhibition of HIV replication.

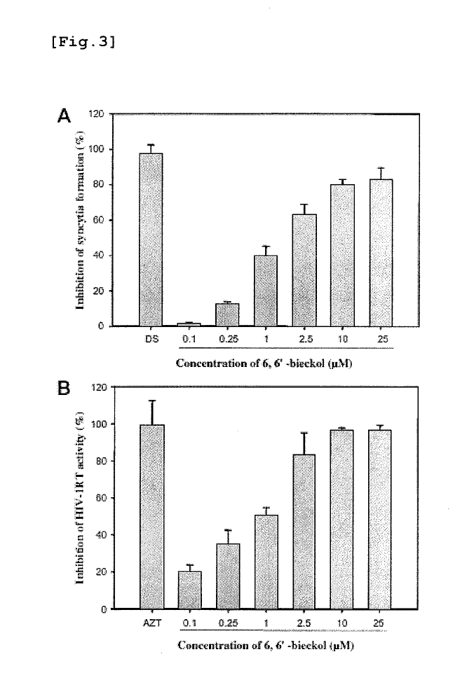

As demonstrated in FIGS. 1B and 3B, data obtained from HIV-1 induced cytopathic effects and RT activity studies indicated that HIV-1 replication inhibition correlated with the inhibition of reverse transcriptase. 6,6′-bieckol inhibited both HIV-1 induced lytic effect and HIV-1 RT activity almost completely. These data indicate that unlike other tannins, 6,6′-bieckol is non-cytotoxic at a concentration inhibiting HIV-1 replication and reverse transcriptase activity effectively.

Entry step of HIV-1 is an attractive target for new generation drug candidates. Therefore, the inhibitory effect of 6,6′-bieckol on HIV-1 entry was investigated. Nonaka et al. claimed that a variety of tannins inhibited the replication of HIV by interfering with HIV-cell interactions. 6,6′-bieckol inhibited the HIV-1 induced syncytia formation as a result of data obtained from co-cultivation of uninfected C8166 cells with H9/HIV-1IIIB cells. Tannins show their inhibitory activity on HIV-1 entry by inhibiting gp41 six-helix bundle formation. However, when entry inhibition profile of 6,6′-bieckol is compared with the inhibition of cytopathic effects, it could be concluded that HIV-1 induced cytopathic effect is reduced not only by the inhibition of entry, but also by subsequent inhibition of HIV-1 reverse transcriptase enzyme activity.

### Example 3-5: p24 ELISA

H9 cells (3×10⁶ cells/ml) were incubated in the presence or absence of HIV-1IIIB for 1 h at 37° C. Cells were washed to remove unbound viruses and resuspended at 3×10⁵ cells/ml in the culture medium. Aliquots of 1 ml were placed in a 24-well culture plate containing an equal volume of medium with/without 6,6′-bieckol. AZT was treated as positive control. In order to determine the amount of virus released to the medium, HIV-1 p24 antigen capture ELISA was carried out with a commercial kit (Perkin-Elmer Life Sciences, Boston, Mass.) according to the manufacturer's instructions.

p24 antigen production was determined with p24 antigen capture ELISA and Western blot analysis. 6,6′-bieckol inhibited the production of p24 antigen more than 90% at 10 μM as supported by data obtained from ELISA (FIG. 1D). As shown in FIG. 2C, the inhibition of p24 antigen production at a concentration of 25 μM of 6,6′-bieckol was comparable to that of AZT (5 μM). 8,8′-bieckol, which is structurally similar to 6,6′-bieckol, inhibited HIV-1 recombinant RT and protease activity with IC₅₀ of 0.51 μM and 81.5 μM, respectively. Reverse transcriptase inhibitory activity of 6,6′-bieckol was consistent with those of 8,8′-bieckol.

According to the Western blot analysis, no or weak bands were detected at the locations of p55 gag related proteins (p55, p41, and p24) in addition to p24, which indicates that 6,6′-bieckol did not inhibit the protease. However, 6,6′-bieckol inhibited the p24 antigen production in a dose-dependent manner both in culture supernatant and cell (FIGS. 2A and 2B) by inhibiting the activity of reverse transcriptase. In is concluded that the structure-activity relationship is based on the unique skeleton with dibenzodioxin linkage, linkage positions of phloroglucinols, and phenolic groups exiting in 6,6′-bieckol.

### Example 3-6: Co-cultivation Assay

3×10⁴ C8166 cells were pre-treated with various concentrations of 6,6′-bieckol for 2 h and co-cultured with 3×10³ H9 cells which are chronically infected with HIV-1IIIB, at 37° C. in a humidified atmosphere of 5% CO₂. After 72 h of incubation, the number of syncytia formed was counted using a microscope.

The fusion of virus or HIV-infected cells with uninfected target cells is a critical step in HIV infection. According to data obtained from co-cultivation of C8166 cells with H9 cells chronically infected with HIV-1IIIB, 6,6′-bieckol inhibites the entry of HIV-1. 6,6′-bieckol inhibited cell-virus fusion (FIG. 1B) and cell-cell fusion (FIG. 3A). Co-cultivation assay showed that with treatment at the highest concentration of 6,6′-bieckol, the inhibition of syncytia formation was around 80%.

1. A HIV-1 inhibiting pharmaceutical composition containing phloroglucinol polymer compounds derived from Ecklonia Cava as an active ingredient.

2. The HIV-1 inhibiting pharmaceutical composition of claim 1, wherein the phloroglucinol polymer compounds derived from Ecklonia Cava is 6,6′-bieckol.
